(19) United States
(12) Patent Application Publication
(10) Pub. No.: US 2013/0139569 A1
(43) Pub. Date: Jun. 6, 2013

(54) COLUMN WITH GAS DISTRIBUTION AND METHOD OF CHARACTERIZING A GAS-LIQUID CONTACTING ELEMENT

(71) Applicants: Pascal Alix, Roussillon (FR); Ludovic Raynal, Oullins (FR)

(72) Inventors: Pascal Alix, Roussillon (FR); Ludovic Raynal, Oullins (FR)

(21) Appl. No.: 13/808,620

(22) PCT Filed: Jun. 29, 2011
(86) PCT No.: PCT/FR2011/000379

§ 371 Date: Feb. 21, 2013

(30) Foreign Application Priority Data
Jul. 8, 2010 (FR) 1002875

(51) Int. Cl. B01F 3/04; G01N 33/00

(52) U.S. Cl. CPC B01F 3/04; G01N 33/0009
USPC 73/31.04; 261/74; 73/23.2

(57) ABSTRACT

The object of the invention is a gas-liquid contacting column comprising a gas supply line (2), a liquid supply line (4), at least one functional zone (6) comprising at least one gas-liquid contacting element, functional zone (6) being arranged between gas supply line (2) and liquid supply line (4). Gas supply line (2) cooperates with a distribution zone (8) arranged between the gas supply line and the functional zone, distribution zone (8) consisting of a packing whose height is so selected that the gas coming from the distribution zone circulates with a local velocity over the bed inlet section of the functional zone ranging between −50% and +50% of the average velocity of the gas circulating in the column.

### FIELD OF THE INVENTION

The present invention relates to the sphere of fluid contacting equipments.

The purpose of contacting columns is to contact fluids so as to achieve material or heat transfers between a gas and a liquid. This type of fluid contacting equipment is widely used to carry out distillation, rectification, absorption, heat exchange, extraction, chemical reaction, etc., operations.

### BACKGROUND OF THE INVENTION

Contacting columns generally consist of a cylindrical enclosure provided with inner contacting elements promoting fluid exchange. In the column, the fluids can circulate in a co-current or counter-current flow. In general, the column enables intimate contact between an ascending gas phase and a descending liquid phase. The contacting elements that increase the contact surface between the fluids can be trays, structured packings, i.e. an orderly arrangement of juxtaposed elements, corrugated sheets for example, or random packings, i.e. anarchistic stockings of elements, rings or spirals for example.

For proper operation of a contacting column, it is important that the gas enters the contacting element homogeneously over the entire diameter of the column.

“Industrial Column” Case

In industrial columns, the problem consists in distributing the gas as homogeneously as possible over very large diameters. These diameters usually range between 1 and 10 meters, they can even reach 12 m in thermal power station fumes desulfurization units, generally between 2 and 8 meters, but they can reach 12 to 15 meters. The fluids therefore have to be well distributed to allow optimum use of the functional zone. Gas distribution is generally achieved by complex and therefore costly distributors. A second constraint relates to the pressure drop generated by the distribution system. In the case of CO₂ capture on industrial fumes available at ambient pressure, a compressor is necessary to overcome the pressure drop induced by the column (inlet and distribution, reaction zone, outlet). It is estimated that the energy penalty induced by an extra pressure drop of about 50 mbar corresponds to a cost of 1.1 M [symbol] a year. It is therefore essential to favour technologies providing good initial distribution for a minimum amount of pressure drop, otherwise the equipments (column diameter, heights associated with the distributor size and the compressor dimensioning) have to be oversized. Of course, technologies meeting these various criteria with a minimum cost are preferably used.

Industrial choices therefore often are a matter of compromise between cost and performances. There are simple and inexpensive solutions in terms of investment for gas distribution, as described for example in U.S. Pat. No. 6,341,765. On the other hand, this type of solution generates high pressure drops and it is very moderately effective. More complex solutions, sometimes very bulky, allow to obtain better results than those described in U.S. Pat. No. 5,106,544 or GB-1,119,699, but they are very expensive to buy and to install.

The present invention allows to meet all the desired criteria:
very high distribution efficiency,
very low pressure drop,
low investment cost with no additional installation cost,
very small size.

“Laboratory Column” Case

Columns of smaller diameter are generally used for characterizing a contacting element in terms of hydrodynamics and mass transfer. This determination is generally performed in a laboratory column of smaller diameter than industrial columns, typically ranging between 0.1 and 1.0 meter in diameter.

Using perforated pipe type distributors (also known as spargers) below the functional zone comprising the gas-liquid contacting elements induces a turbulence zone directly below the functional zone and it disturbs the determination of the flooding factor when columns with a diameter less than or equal to 1 meter are used. Thus, packing manufacturers usually recommend, in the case of columns less than 1 meter in diameter, a lateral gas inlet followed by a stilling zone over a height of at least 0.5 meters, or even several meters.

Although this configuration at the column inlet allows good determination of the flooding factor, it is a problem for determining transfer coefficients. Indeed, it generates a high turbulence zone below the functional zone, i.e. the zone comprising the packing and providing gas-liquid contacting, and wherein significant unwanted inlet effects are generated.

Furthermore, the lineic pressure drop of a gas flowing through a packing is low, of the order of 1 to 2 mbar/m. To obtain good determination of the industrial column dimensioning criteria, it is therefore essential, considering the low pressure drop at the inlet, to have a gas flow distribution as homogeneous as possible at the inlet.

The present invention aims to use a structured or random packing height for homogenizing the gas flow over the diameter of the column so as to optimize the operation of an industrial column or to overcome inlet effects in a characterization column in order to use the measurements for extrapolation to the industrial scale.

### SUMMARY OF THE INVENTION

In general terms, the object of the present invention is a gas-liquid contacting column comprising a gas supply line, a liquid supply line, at least one functional zone comprising at least one gas-liquid contacting element, the functional zone being arranged between the gas supply line and the liquid supply line, characterized in that the gas supply line cooperates with a distribution zone arranged between the gas supply line and the functional zone, the distribution zone consisting of a packing whose height is so selected that the gas coming from the distribution zone circulates with a local velocity ranging between −50% and +50% over the column section at the functional zone inlet in relation to the average velocity of the gas circulating in the column.

According to the invention, the flooding factor of the packing of the distribution zone can be at least 20% less than the flooding factor of the contacting elements of the functional zone. To make this comparison, the flooding factors can be determined by counter-current contacting of liquid water and air in the packing considered, the water flow rate ranging between 5 and 150 m³/m²/h, preferably between 20 and 60 m³/m²/h.

The height of the distribution zone can range between 0.05 and 2.0 m.

A space can be provided between the distribution zone and the functional zone, the space being at least above 50 mm in height.

The gas supply line can be oriented in a lateral direction, i.e. perpendicular to the height of the column.

The packing of the distribution zone can be made of metal, of a polymer material or of ceramic.

The void fraction of the distribution zone packing can range between 0.90 and 0.99, and the geometric surface area of said packing ranges between 80 and 750 m²/m³.

Another object of the present invention is a method of characterizing a gas-liquid contacting element, wherein the following stages are carried out:

a) arranging said contacting element in a column comprising a gas supply line and a liquid supply line, the contacting element being arranged between the gas supply line and the liquid supply line,

b) providing a gas distribution zone in the column between the gas supply line and said contacting element, the distribution zone consisting of a packing,

c) performing at least one measurement on the gas circulating between the distribution zone and said element.

According to the invention, the measurement performed in stage c) can be used to determine at least one characteristic of said contacting element: flooding curve, liquid side transfer coefficient, gas side transfer coefficient, effective surface area.

For example:
the flooding curve is determined through pressure measurement,
the effective surface area is measured by CO₂ absorption by a soda or amine solution,
the liquid side transfer coefficient is determined by chemical absorption of CO₂ in a carbonate or amine solution,
the gas side transfer coefficient is determined by chemical absorption of SO₂ in a soda solution or by water evaporation in a non-water saturated gas.

In the column according to the invention, good homogenization of the gas flow at the functional zone inlet is obtained, for a wide range of column diameters. The invention is well suited for industrial-size columns but it can also be advantageously used in laboratory columns for characterizing the hydrodynamics and the material transfer of the functional zone. In particular, implementing a column according to the invention allows to substantially improve the quality of the transfer coefficient measurements by minimizing the inlet effects without distorting the flooding measurement.

### BRIEF DESCRIPTION OF THE FIGURES

Other features and advantages of the invention will be clear from reading the description hereafter, with reference to the accompanying figures wherein:

FIG. 1 diagrammatically shows a column according to the invention,

FIG. 2 diagrammatically shows a laboratory column according to the invention,

FIG. 3 diagrammatically shows a column used for the comparative examples,

FIG. 4 shows various gas distributors in the case of an 8-m diameter column,

FIG. 5 shows the gas distributions obtained with the gas distributors of FIG. 4 in the case of an industrial column,

FIG. 6 shows a comparison of the variation in lineic pressure drop ΔP/L as a function of the gas flow rate factor Fₛ depending on the distributor used (FIG. 4),

FIG. 7 shows the gas distributions obtained with gas distributors of FIG. 4 in the case of a laboratory column.

### DETAILED DESCRIPTION

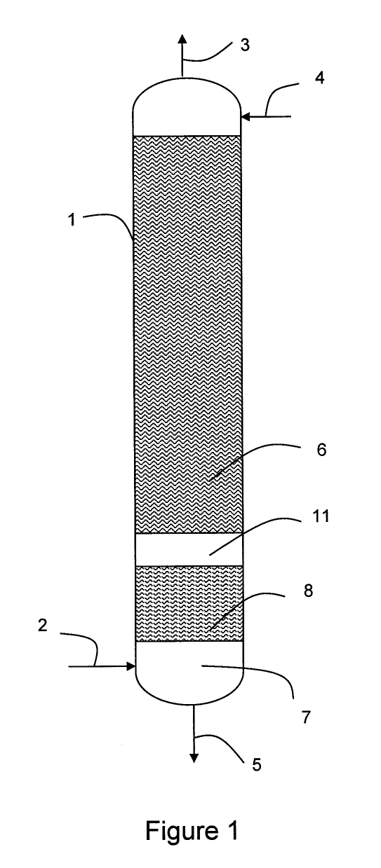

FIG. 1 shows a column (1) comprising a functional zone (6) provided with gas-liquid contacting elements.

The column allows intimate contact, in the functional zone, between an ascending gas phase and a descending liquid phase. The contacting elements that increase the contact surface between the fluids can be structured packings, i.e. an orderly arrangement of juxtaposed elements, corrugated sheets for example, or random packings, i.e. anarchistic stackings of elements, rings or spirals for example.

Structured packings can consist of folded sheets arranged in an organized manner as big blocks, as described for example in documents U.S. Pat. No. 3,679,537 and U.S. Pat. No. 4,296,050 (Mellapak type packings marketed by Sulzer Chemtech). The new generation of random packings generally consists of metal elements provided with perforations and arc portions with sophisticated shapes, such as the IMTP packings marketed by Koch Glitsch.

The gas is injected laterally through line (2) below contacting elements (6). The liquid is injected into column (1) through line (4) above contacting elements (6). The gas, after contacting the liquid in packing (6), is discharged through a line (3) at the top of the column and the liquid through a line (5) at the bottom of the column.

According to the invention, a gas distribution zone (8) consisting of a structured packing or of a random packing bed is arranged in column (1). The distribution zone is positioned between the injection point where the gas is injected through line (2) into column (1) and contacting element (6). Thus, the gas injected into column (1) flows through packing (8) prior to reaching contacting elements (6). The purpose of packing (8) is to homogenize distribution of the gas flow over the section of column (1), notably by homogenizing the rate of circulation of the gas over the section of column (1). For example, according to the invention, packing (8), and notably the height thereof, is so selected that the gas flowing from packing (8) has a local velocity over the column section at the functional zone inlet ranging between −50% and +50% of the average gas velocity, preferably between −30% and +30% of the average gas velocity. The average velocity corresponds to the total gas flow rate divided by the column section at the outlet of packing (8), i.e. in the upper part of packing (8).

The structured or random packings that distribution zone (8) is equipped with can be made of metal, of a polymer material or of ceramic, preferably metal.

The void fraction of the packing of distribution zone (8) can range between 0.60 and 0.99, preferably between 0.90 and 0.99. The geometric surface area of said packing can range between 80 and 750 m²/m³, preferably between 80 and 250 m²/m³.

For example, distribution zone (8) comprises a pile of 1 to 5 structured packing plates, preferably 1 to 3 plates, or a random packing bed. A structured packing plate is understood to be a packing block of height ranging between 180 and 250 mm. The height of distribution zone (8) can range between 0.05 and 2.0 m, preferably between 0.2 and 0.7 m. An excellent height value ranges between 0.4 and 0.6 m. A small packing height in zone (8) is sufficient for homogeneous gas distribution over the column section. This small packing height only generates a very low pressure drop. Gas distribution zone (8) according to the invention therefore allows to homogenize the rate of circulation of the gas over the entire column section while limiting pressure drops.

Distribution zone (8) is arranged in the lower part of column (1), below (or upstream from, when following the path followed by the gas) functional zone (6). Distribution zone (8) and functional zone (6) can be either apart (therefore separated by a space (11) as shown in FIG. 1), or next to one another (no space (11) in this case). In the case of laboratory columns, the two zones can be apart so as to install a pressure detector and/or a branch connection for withdrawing a gas or a liquid portion for measurements just at the functional zone inlet.

Preferably, the type of structured or random packing used in distribution zone (8) can be so selected as to be more capacitive than the structured or random packing used in the functional zone. It is more capacitive, which means that the packing of distribution zone (8) preferably reaches its flooding point for a gas flow rate that is 20% to 50% higher than that of functional zone (6), so as to limit any early flooding initiated in distribution zone (8) as a result of uncontrolled inlet effects. This comparison of the flooding point of the packing of distribution zone (8) in relation to that of functional zone (6) is made for the passage of one and the same gas, air for example, and the passage of one and the same liquid, water for example. The gas flow rate at the flooding point can be determined for liquid flow rate ranges between 5 and 150 m³/m²/h, preferably between 20 and 60 m³/m²/h. These values have to be adjusted depending on the desired application. For example, in the case of fume scrubbing with an aqueous solution comprising 30 wt. % MonoEthanolAmine, the gas flow rate at the flooding point can be determined for a liquid flow rate of 30 m³/m²/h. For gas purification, the gas flow rate at the flooding point can be determined for a liquid flow rate of 10 m³/m²/h. For washing a gas with a high acid gas content, the gas flow rate at the flooding point can be determined for a liquid flow rate of 100 m³/m²/h.

The column according to the invention can be used for natural gas deacidizing, fumes decarbonation or Claus tail gas treatment, or in any type of gas treatment. In these applications, the gas to be treated is contacted with a liquid absorbent solution in a column provided with a gas distribution zone consisting of a packing.

The distribution zone can also be used in a laboratory column for determining various characteristics of a gas-liquid contacting element.

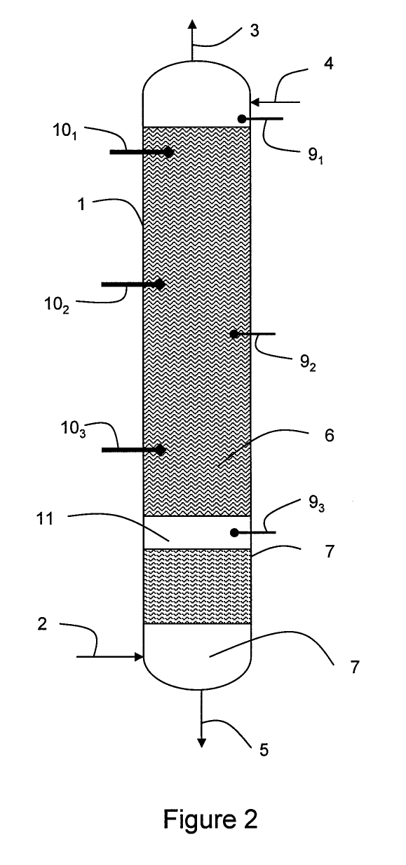

FIG. 2 shows a laboratory column with a distribution zone (8) according to the invention. The reference numbers of FIG. 2 identical to those of FIG. 1 designate the same elements.

Distribution zone (8) according to the invention, i.e. consisting of a packing, allows to homogenize the gas stream entering the gas-liquid contacting element (6) studied, and thus to obtain measurements from detectors (9₁, 9₂, 9₃) and samples from branch connections (10₁, 10₂, 10₃) that correspond to a homogeneous operation over the section of the element (6) studied.

The purpose of characterization is, among other things, to determine the transfer and mass performances, and in particular the effective surface area ae, as well as the performances in terms of pressure drop and in particular the flooding factor Fc. Effective surface area ae corresponds to the surface area really available for gas-liquid contacting in the packing. Flooding factor Fc is the ratio of the gas flow rate circulating through the packing to the gas flow rate corresponding to the flooding limit for one and the same liquid flow rate. Flooding corresponds to the operating limit of the contacting column provided with a packing, i.e. to the maximum gas flow rate that can be passed into the column for a constant liquid flow rate in the case of a counter-current flow.

It is possible to establish packing flooding curves of good reliability, which amounts to determining, for a fixed liquid flow rate, the allowable maximum gas flow rate. Determining flooding curves is well known to the person skilled in the art and it generally consists in measuring, for a fixed liquid flow rate, the pressure drop of the gas flowing through packing bed (6) for different gas flow rates. The pressure drop is measured by means of pressure detectors arranged at the functional zone inlet and outlet, and/or all along the functional zone (detectors 9₁, 9₂, 9₃ in FIG. 2). The flooding curves are then used to calculate the diameter of industrial columns. The diameter of the characterization column is set so as to overcome size effects as much as possible. In general, it consists in keeping a minimum ratio between the column diameter and the characteristic dimension of the packing tested; the latter can be, non-exhaustively, a diameter, a length or the equivalent diameter of a sphere of same density. The characteristic dimension of a structured packing can be, for example and non-exhaustively, the size (or fold) of a corrugation or the hydraulic diameter of a channel (which corresponds to 4× the welted perimeter to the surface area of flow, i.e. 4/ag for a completely wet structured packing, ag being the geometric surface area of the packing). In general, taking account of the characteristic size selected and of the minimum ratio between the column diameter and the characteristic dimension of the packing tested leads to testing column diameters ranging between 0.10 and 1.0 m, preferably between 0.4 and 1.0 m. The height of functional zone (6) can be fixed by the person skilled in the art as it depends on the chemical system used. In general, it ranges between 0.5 and 5.0 m, preferably between 2.0 and 5.0 m.

The gas flow rate factor, Fₛ=\sqrt{ρ_{g}}, V_{sg} (with ρ_{g}: gas density in kg/m³ and V_{sg}: gas surface velocity in m·s⁻¹), generally ranges between 0.2 and 5 Pa^{0.5}, preferably between 0.5 and 4.0 Pa^{0.5}.

The liquid flow rate generally ranges between 1 and 200 m³/m²/h, preferably between 5 and 100 m³/m²/h.

The method according to the invention also allows to determine the transfer parameters: gas and liquid side transfer coefficients, k_{L}, k_{G}, and the gas-liquid interfacial surface area ae. The flow of a compound A is therefore measured from the gas phase to the liquid phase, or from the liquid phase to the gas phase.

The interfacial surface area (or effective surface area ae) can be measured by chemical absorption of a gas by a liquid, for example by CO₂ absorption by a soda solution, or by CO₂ absorption by an amine solution such as MEA or DEA.

The liquid side transfer coefficient (k_{L}) can be determined by physical absorption of ammonia in water, or by chemical absorption of CO₂ in a carbonate solution or in an amine solution of MDEA type.

The gas side transfer coefficient (k_{G}) can be determined by chemical absorption of SO₂ in a soda solution, or by water evaporation in a non-water vapour saturated gas.

Of course, there are many other systems in the literature, such as, for example, chemical absorption of NH₃ in a H₂SO₄ solution, physical absorption of SO₂ in water, O₂ desorption from water, etc.

The transfer coefficients are determined by taking liquid and gas samples. The gas and liquid samples are generally taken at the functional zone inlet and outlet, and/or all along the functional zone, i.e. by taking samples at branch connections 10₁, 10₂, 10₃ in FIG. 2.

The liquid phase can be analyzed by potentiometry, chromatography, Raman spectrometry, or any other technique known to the person skilled in the art.

The gas phase can be analyzed by chromatography, infrared spectrometry or any other gas analysis technique known to the person skilled in the art.

Compound A is generally selected from the group made up of air, NH₃, H₂O, CO₂, SO₂, N₂, O₂, H₂S, NOₓ, SOₓ, COS, RSH, preferably from the group made up of air, NH₃, H₂O, CO₂, SO₂, O₂, H₂S.

The height of distribution zone (8) can range between 0.05 and 1.0 m, preferably between 0.2 and 0.5 m.

Distribution zone (8) can be arranged in the lower part of the column, below functional zone (6). Preferably, the two zones are apart. In the case of laboratory columns, the height of space (11) between distribution zone (8) and functional zone (6) can be at least 50 mm, preferably at least 100 mm. It is thus possible to provide a pressure detector and/or a branch connection (9₃) to perform a measurement or to take a sample between functional zone (6) and distribution zone (8).

The method according to the invention is particularly well suited for acquisition of experimental data that can subsequently be integrated into simulators allowing dimensioning of (reactive or not) distillation and reactive absorption units.

### EXAMPLES

The advantages of the invention are illustrated by the comparative examples below. The examples are based on numerical flow simulation computations (CFD Computational Fluid Dynamics) carried out with the commercial software Fluent 6.3.26. The goal is to compare the quality of the distribution obtained and the associated pressure drop between different distribution system geometries.

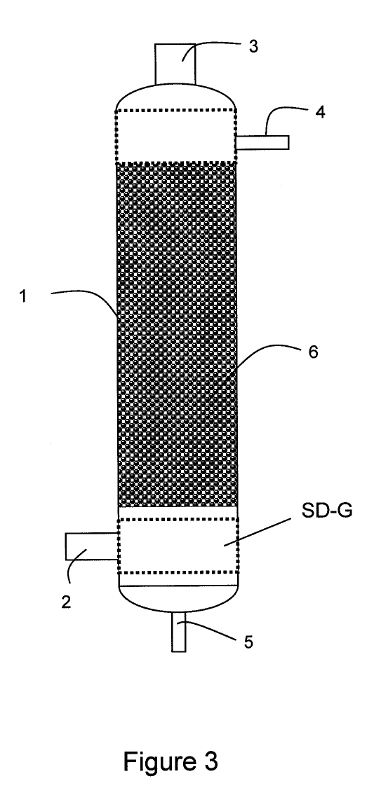

FIG. 3 shows a simplified column diagram with the gas distribution system (SD-G). The reference numbers of FIG. 3 identical to those of FIG. 1 designate the same elements.

### Example 1

### Case of an Industrial Column

In the present case, an 8-m diameter column (1) is equipped with a random packing bed corresponding to IMTP-40. The mass flow rate of the gas flowing in through line (2) is 121 kg/s.

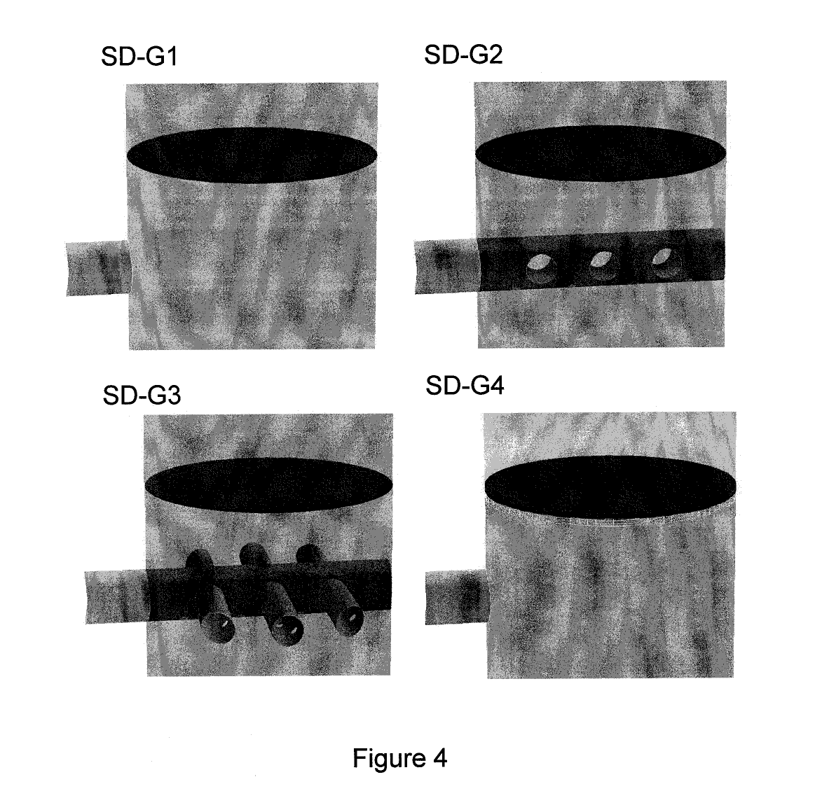

FIG. 4 shows four different gas distribution configurations (SD-G):
SD-G1: no distribution system, a case often selected for small-size columns,
SD-G2: pipe distributor,
SD-G3: comb distributor, a commonly selected geometry for the industrial scale,
SD-G4: no distributor but two structured packing plates positioned just below the packing bed, a geometry corresponding to the present invention. The two plates, nearly 200 mm in height, of structured packing corresponding to Mellapak 250X are offset by 90° in relation to each other as regards the direction of the plates of the structured packing.

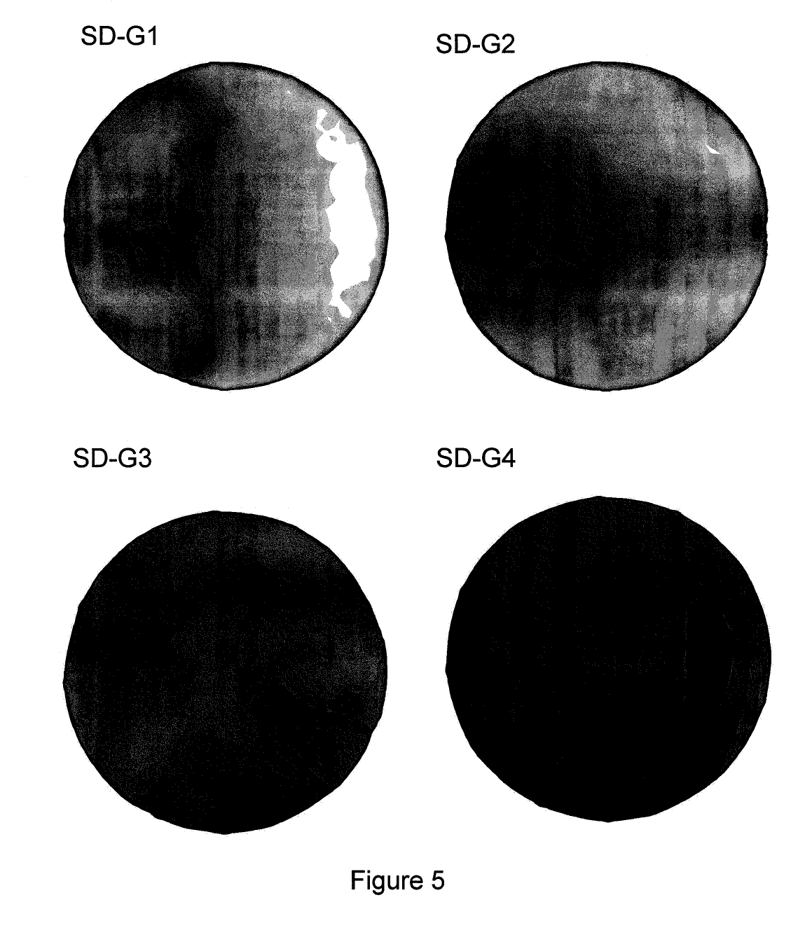

FIG. 5 shows the maps, or contours, of the amplitude of the local gas velocities at the packing bed inlet for the various systems studied. The velocity range is identical in the four cases, between 1.5 m/s in black and 8 m/s in white.

The quantitative results related to this example are given in Table 1. This table shows, for each of the four systems tested, the values of the velocity amplitudes U of the three components of the velocity vectors (Ux, Uy, Uz) in directions x, horizontal corresponding to the axis of the gas inlet tube, y, horizontal perpendicular to x, and z, vertical oriented upwards. The pressure drop values associated with the lost energy required to provide the distribution are also given. Finally, the standard deviation values of the velocities at the packing bed inlet (deviation from ideal distribution) are also given. In an ideal distribution case, components Ux and Uy should be zero, and component Uz should be equal to the discharge velocity, which is here 1.8 m/s.

TABLE 1

| | U (m/s) | | Ux (m/s) | | Uy (m/s) | | Uz (m/s) | | DP | deviation/ideal |
|---|---|---|---|---|---|---|---|---|---|---|
| System | min | max | min | max | min | max | min | max | (mbar) | (%) |
| SD-G1 | 0 | 10.7 | −10.3 | 2.9 | −3.6 | 3.4 | 1.4 | 3.7 | 104 | 269 |
| SD-G2 | 1.8 | 8.2 | −5.8 | 1.3 | −7.0 | 7.0 | 1.6 | 3.3 | 108 | 238 |
| SD-G3 | 1.9 | 4.0 | −2.2 | 2.5 | −3.0 | 2.5 | 1.8 | 2.4 | 200 | 100 |
| SD-G4 | 1.7 | 2.7 | −0.3 | 0.1 | −0.2 | 0.2 | 1.7 | 2.7 | 106 | 21 |

Significant differences can be observed between the four geometries in FIG. 5. The first image shows that, without a distribution system (SD-G1), a very high velocity heterogeneity (translated into the colour contrasts) can be seen, which is harmful to the proper functioning of the column. Installing a conventional system of perforated pipe type (SD-G2) does not allow to significantly reduce the heterogeneities observed. A more complex system, of comb type (SD-G3), allows to obtain a greatly improved distribution, the heterogeneities have decreased significantly (the maximum velocity is not more than 2.2 times the discharge velocity, whereas the latter reached values close to 5 in the two previous cases—see Table 1). This improvement has a cost, since it involves a greatly increasing pressure drop, from nearly 100 mbar to nearly twice as much. In the case corresponding to the present invention (SD-G4), an improvement in the distribution quality can be noted, as well as a reduction in the associated pressure drop. In fact, the heterogeneities are reduced by a factor 5 in relation to the previous case, the maximum velocity value is not more than 1.5 times that of the discharge velocity, and the horizontal velocities have decreased by a factor 10. Finally, it is observed that the associated pressure drop is reduced by a factor 2 in relation to the case with a complex system (SD-G3) for a widely improved result quality; it is noted that the same pressure drop values as in the case without a distribution system are obtained, it therefore seems to be difficult to decrease this value any further.

An excellent result in terms of deviation from the ideal distribution in relation to the required pressure drop is thus obtained according to the invention.

### Example 2

### Case of a Laboratory Column: Determining the Flooding Limit

We want to determine the flooding limit of a commercial metal structured packing.

A 150-mm diameter column according to the layout of FIG. 3, operating under counter-current water and air flow conditions, is used according to different configurations. A liquid stream is injected through line 4 at 50 m³/h, and a gas stream of variable flow rate is injected through line (2). In functional zone (6), the packing studied is the MellapakPlus 252.Y (Sulzer Chemtech). 9 packing plates are arranged in zone (6), which corresponds to a height of approximately 1.9 m.

Configuration 1 (in accordance with the invention, corresponds to case SD-G4 of FIG. 4) shows a lateral gas inlet, then a distribution zone consisting of 2 structured packing plates, i.e. a height of approximately 0.4 m. The space between the distribution zone and the functional zone is around 0.1 m. Determination of the pressure drop is performed between the inlet and the outlet of functional zone (6).

Configuration 2 (not in accordance with the invention, corresponds to case SD-G1 of FIG. 4) shows a lateral gas inlet with no distribution zone, the pressure drop being determined at the inlet and the outlet of the bed consisting of 9 structured packing plates.

Configuration 3 (not in accordance with the invention, corresponds to case SD-G3 of FIG. 4) uses a sparger type gas distributor and the pressure drop is measured at the inlet and the outlet of the bed consisting of 9 structured packing plates.

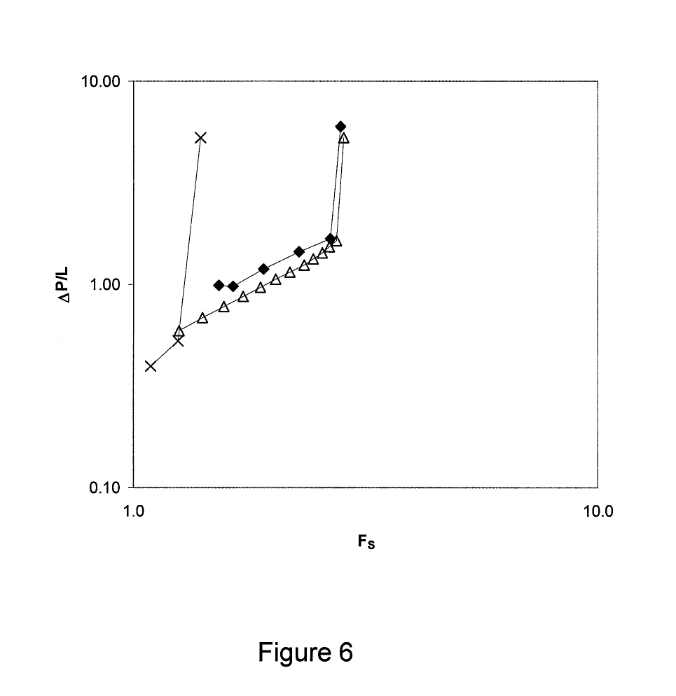

FIG. 6 shows the variation in lineic pressure drop ΔP/L as a function of the gas flow rate factor Fₛ. The lineic pressure drop ΔP/L is expressed in mbar/m. Gas flow rate factor Fₛ corresponds to the square root of the gas density ρ_{g} multiplied by the superficial gas velocity Vs (Fs=\sqrt{ρ_{g}}×Vs) and it is expressed in Pa^{0.5}. The flow rate factor variation for the various configurations leads to the results given in FIG. 6 (♦: configuration 1, Δ: configuration 2, x: configuration 3). The various curves are obtained by measuring the pressure drop of the gas between the inlet and the outlet for different gas flow rate values.

Thus, configurations 1 and 2 (♦ and Δ) lead to similar results, whereas the sparger type distributor (configuration 3 (x)) considerably modifies the flooding limit. Therefore, using configuration 1 according to the invention allows not to modify the flooding limit of the packing studied.

### Example 3

### Case of a Laboratory Column: CFD Computations

Numerical simulations have been carried out by taking into account the column of Example 2 in order to illustrate the distribution differences between various distribution zones at the column inlet.

The distribution zones selected are configurations SD-G1, SD-G3 and SD-G4 of FIG. 4 in the column of FIG. 3.

The gas flow rate selected is 2.0 m/s. The inlet of the functional zone bed is approximately 1 meter above the injection zone.

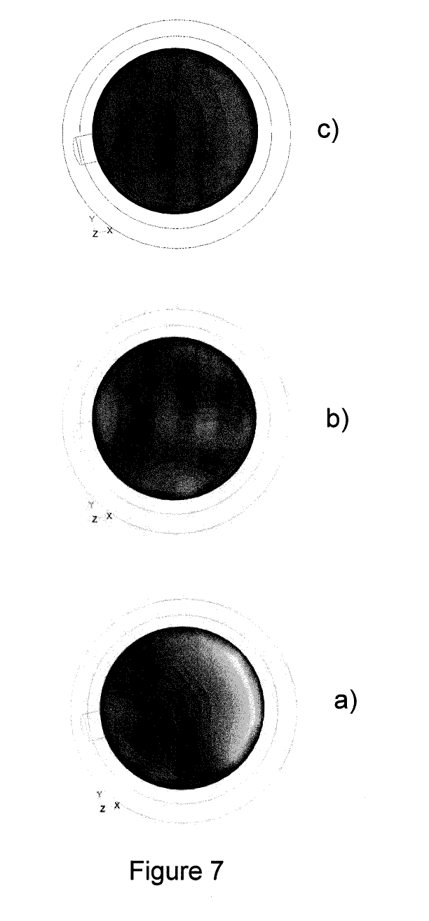

In FIG. 7, representation a) corresponds to the configuration without a distributor (SD-G1), representation b) corresponds to the configuration with a sparger type distributor (SD-G3) and representation c) corresponds to the configuration with a distribution zone according to the invention (SD-G4).

As regards the colour range, black corresponds to a 0 m/s velocity and white to an 11 m/s velocity.

It can be observed in FIG. 7 that the most homogeneous gas distribution is obtained with the column according to the invention, i.e. with a gas distribution zone consisting of a packing.

Table 2 below allows to quantify the distribution differences according to the various configurations.

TABLE 2

| | without a | with a | with a |
|---|---|---|---|
| | distributor | distributor | distribution zone |
| | configuration | configuration | configuration |
| | SD-G1 | SD-G3 | SD-G4 |
| max bed inlet velocity | 11 | 5 | 3 |
| (m/s) | | | |
| standard deviation of bed | 123 | 37 | 20 |
| inlet velocities (%) | | | |
| pressure drop (mbar) | 0.5 | 95.5 | 0.5 |

Table 2 clearly shows that the configuration with a distribution zone leads to the best results, whether in terms of distribution homogeneity or of pressure drop.

1) A gas-liquid contacting column comprising a gas supply line, a liquid supply line, at least one functional zone comprising at least one gas-liquid contacting element, functional zone being arranged between gas supply line and liquid supply line, characterized in that gas supply line cooperates with a distribution zone arranged between gas supply line and functional zone, distribution zone consisting of a packing whose height is so selected that the gas coming from distribution zone circulates with a local velocity ranging between −50% and +50% of the column section at the inlet of functional zone in relation to the average velocity of the gas circulating in the column.

2) A column as claimed in claim 1, wherein the flooding factor of the packing of distribution zone is at least 20% less than the flooding factor of the contacting elements of functional zone.

3) A column as claimed in claim 1, wherein the height of distribution zone ranges between 0.05 and 2.0 m.

4) A column as claimed in claim 1, characterized in that a space is provided between distribution zone and functional zone, space being at least above 50 mm in height.

5) A column as claimed in claim 1, wherein gas supply line is oriented in a perpendicular direction with respect to the height of the column.

6) A column as claimed in claim 1, wherein the packing of distribution zone is made of metal, of a polymer material or of ceramic.

7) A column as claimed in claim 1, wherein the void fraction of the packing of distribution zone ranges between 0.90 and 0.99, and the geometric surface area of said packing ranges between 80 and 750 m²/m³.

8) A method of characterizing a gas-liquid contacting element wherein the following stages are carried out:
a) arranging said contacting element in a column comprising a gas supply line and a liquid supply line, the contacting element being arranged between the gas supply line and the liquid supply line,
b) providing a gas distribution zone in the column between the gas supply line and said contacting element, distribution zone consisting of a packing,
c) performing at least one measurement on the gas circulating between the distribution zone and said element.

9) A method as claimed in claim 8, wherein the measurement performed in stage c) is used to determine at least one characteristic of said contacting element: flooding curve, liquid side transfer coefficient, gas side transfer coefficient, effective surface area.

10) A method as claimed in claim 9, wherein:
the flooding curve is determined through pressure measurement,
the effective surface area is measured by CO₂ absorption by a soda or amine solution,
the liquid side transfer coefficient is determined by chemical absorption of CO₂ in a carbonate or amine solution,
the gas side transfer coefficient is determined by chemical absorption of SO₂ in a soda solution or by water evaporation in a non-water saturated gas.
